(19) United States
(12) Patent Application Publication
(10) Pub. No.: US 2026/0116898 A1
(43) Pub. Date: Apr. 30, 2026

(54) Preparation Method and Application of Imidazole-Functionalized Metal-Organic Framework Material Im RE-MOF With Lipase-Mimetic Catalytic Activity

(71) Applicant: Jiangnan University, Wuxi (CN)

(72) Inventors: Feng Wang, Wuxi (CN); Tian Qiu, Wuxi (CN); JiaQi Lu, Wuxi (CN); JinYu Cui, Wuxi (CN); Mengjie Zhang, Wuxi (CN)

(21) Appl. No.: 19/426,225

(22) Filed: Dec. 19, 2025

(30) Foreign Application Priority Data
Aug. 27, 2025 (CN) 202511206360.1

(51) Int. Cl. C07F 7/00; B01J 31/02; B01J 31/16; C07C 201/12; C07D 207/16; C07D 211/60; C07D 233/64; C07F 3/00; C07F 5/00

(52) U.S. Cl. CPC C07F 7/003; B01J 31/0244; B01J 31/1691; C07C 201/12; C07D 207/16; C07D 211/60; C07D 233/64; C07F 3/003; C07F 5/003

(57) ABSTRACT

Disclosed is a preparation method and application of an imidazole-functionalized metal-organic framework material, Im@RE-MOF, with a lipase-mimetic catalytic activity. The method introduces an imidazole group into a benzene tricarboxylic acid framework through acyl chlorination and Friedel-Crafts acylation reactions to prepare an imidazole-functionalized organic ligand, and the functionalized ligand is self-assembled with a rare earth or transition metal ion (RE=Zr, Sc, or Cd) by a solvothermal method to obtain a target product Im@RE-MOF. The enzyme-mimetic material effectively mimics the catalytic center of natural lipase due to the regular pore structure of the MOF and the catalytic activity of the imidazole group. The material has a stable structure, high catalytic activity, and easy separation and recovery, can catalyze hydrolysis and synthesis reactions of carboxylic esters under mild conditions; overcomes limitations of poor stability and difficulty in recovery of natural lipase; and has broad application prospects in the fields of green chemical industry and organic synthesis.

### TECHNICAL FIELD

The present disclosure relates to a method for preparing a biomimetic material, and in particular to a preparation method and application of an imidazole-functionalized metal-organic framework material, Im@RE-MOF, with a lipase-mimetic catalytic activity.

### BACKGROUND

Lipase (EC 3.1.1.3), as a crucial class of biocatalysts, plays an indispensable role in modern industrial production. Lipase has the most notable advantage of excellent catalytic performance and can catalyze hydrolysis and synthesis reactions of various esters with high chemical selectivity, regioselectivity, and enantioselectivity under extremely mild conditions of normal temperature, normal pressure, and neutral pH. This enables lipase to exhibit tremendous use value in many fields, such as the food industry, pharmaceutical industry, bioenergy, and daily chemical industry, and fully align with a core concept of a contemporary chemical industry's pursuit of “green chemistry” and sustainable development.

Although natural lipase has numerous advantages, inherent properties of natural lipase derived from proteins have also posed insurmountable technical bottlenecks and severely restrict large-scale industrial application of natural lipase. Firstly, the catalytic activity of lipase is highly dependent on a precise three-dimensional spatial structure thereof, and such a structure is extremely sensitive to changes in an external environment. A slight increase in temperature, deviation in pH value, presence of an organic solvent, or mechanical shear force may all lead to irreversible denaturation and inactivation. Secondly, most natural lipases exist in a free state in solvents, making it difficult to effectively separate and recover the natural lipases from a complex reaction system after a reaction is completed. This results in high production costs and may cause product contamination.

To overcome the aforementioned drawbacks, researchers have developed enzyme immobilization technology. Although immobilization has improved stability of an enzyme to some extent and enabled reuse, the technology itself also has problems in that, for example, a preparation process may lead to partial loss of enzyme activity, carriers may leak, and significant mass transfer limitations may reduce apparent catalytic efficiency. Therefore, it has become a highly challenging and crucial frontier direction in the field of catalysis science to fundamentally develop a fully synthetic enzyme-mimetic catalyst, namely “a biomimetic enzyme” or “an artificial enzyme”, that can completely replace natural enzymes and possess high activity, high stability, and easy recovery characteristics.

Metal-Organic Frameworks (MOFs), as a class of emerging crystalline porous materials, provide an ideal platform for constructing high-performance biomimetic enzymes. For example, Liu et al. reported in 2021 that imidazole and a derivative thereof were incorporated into pores of MOF-808 for rapid degradation of a nerve agent, dimethyl 4-nitrophenyl phosphate (ACS Catal. 2021, 11, 3, 1424-1429. 10.1021/acscatal.0c04565). Imidazole was introduced by evaporation and physically loaded onto a surface of MOF-808. An interaction between the imidazole and the MOF is non-covalent, with unstable bonding, making imidazole prone to detachment during the catalysis process. So far, there have been no reports on the use of imidazole-functionalized MOFs for catalyzing the conversion reaction of carboxylic esters.

### SUMMARY

In view of the problems existing in the prior art, the present disclosure provides a preparation method of an imidazole-functionalized metal-organic framework material, Im@RE-MOF, with a lipase-mimetic catalytic activity. A material prepared by the method has a stable structure, can effectively prevent detachment of imidazole groups, and requires mild catalytic conditions.

Technical solution: The present disclosure provides a preparation method for an imidazole-functionalized metal-organic framework material Im@RE-MOF with lipase-mimetic catalytic activity, which includes the following steps:
(1) synthesizing imidazole-4-acetyl chloride (2-(1H-imidazol-5-yl) acetyl chloride) from imidazole-4-acetic acid and a chlorinating agent as raw materials;
(2) carrying out a Friedel-Crafts acylation reaction on the imidazole-4-acetyl chloride and mesitylene as raw materials, with aluminum chloride as a catalyst, to obtain an imidazole ring-modified mesitylene intermediate;
(3) oxidizing all methyl groups of the imidazole ring-modified mesitylene intermediate to carboxyl groups with an oxidant to obtain an imidazole-functionalized benzene tricarboxylic acid ligand; and
(4) carrying out a solvothermal reaction on the imidazole-functionalized benzene tricarboxylic acid ligand and a nodal metal to obtain the imidazole-functionalized metal-organic framework material Im@RE-MOF.

Preferably, the nodal metal is at least one of Zr, Sc, and Cd.

Preferably, in step (1), the chlorinating agent is thionyl chloride or phosphorus trichloride, a molar ratio of the imidazole-4-acetic acid to the chlorinating agent is 1:(0.8-1.1), a reaction solvent is anhydrous dichloromethane with a moisture content of less than 0.01%, and a reaction temperature is 0° C.-5° C.

Preferably, in step (1), a ratio of the imidazole-4-acetyl chloride to the anhydrous dichloromethane is 1 mol: (9-11) mL. After the imidazole-4-acetic acid is dissolved in the dichloromethane, the chlorinating agent is slowly added dropwise, with addition time controlled at 30-60 min. After the dropwise addition is completed, the reaction is continued under stirring for 2-3 h. The purpose of slow dropwise addition is to avoid side reactions, and after the dropwise addition is completed, the reaction is continued for a period of time to ensure complete reaction. After the reaction is completed, the solvent is removed by rotary evaporation. A temperature for rotary evaporation is to be controlled at 35° C.-40° C. to avoid degradation of the acyl chloride. The residue from the rotary evaporation is a crude imidazole-4-acetyl chloride, and the crude product can be directly used in the next reaction step without further treatment.

Preferably, in step (2), a molar ratio of the imidazole-4-acetyl chloride to the mesitylene is 1:(0.8-1.1), an amount of the catalyst used is 1.5 times the molar number of the imidazole-4-acetyl chloride, a reaction solvent is anhydrous dichloromethane, and a reaction temperature is 0° C.-5° C. The mesitylene and the catalyst are dissolved in the dichloromethane, and an anhydrous dichloromethane solution of the imidazole-4-acetyl chloride is slowly added dropwise, with the addition time controlled to be 30-60 min to ensure uniformity of the reaction. After the dropwise addition is completed, the reaction is continued under stirring for 4 hours to 6 hours to allow acyl groups to fully activate and attack benzene rings of the mesitylene to complete the acylation reaction. After the reaction is completed, a reaction system is quenched with a dilute hydrochloric acid solution (with a concentration of about 1 mol/L), and an organic phase is extracted after layering. The extracted organic phase needs to be dried with anhydrous magnesium sulfate and filtered, and then the solvent is removed by rotary evaporation to obtain an imidazole ring-modified mesitylene intermediate product (as shown in formula I):

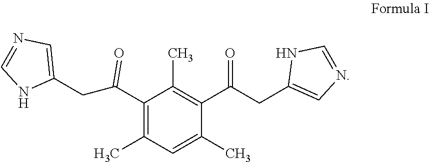 [SMILES: Cc1cc(C)c(C(=O)Cc2cnc[nH]2)c(C)c1C(=O)Cc1cnc[nH]1]

The name of the imidazole ring-modified mesitylene intermediate product is 1,1′-(2,4,6-trimethyl-1,3-phenylene)bis(2-(1H-imidazol-5-yl)ethan-1-one).

Preferably, in step (3), the oxidant is potassium permanganate, a reaction solvent is nitric acid (70%), a temperature for oxidation is 0° C.-10° C., and reaction time is 6-18 h. An amount of the oxidant potassium permanganate used is 2 g to 6 g, and an amount of the reaction solvent used is 100 ml to 300 mL.

Preferably, an oxidation product is purified by column chromatography to obtain a high-purity imidazole-functionalized benzene tricarboxylic acid ligand. A silica gel column used for purification has a particle size of 60-200 meshes, and an eluent is a mixed solvent of n-hexane and ethyl acetate in a volume ratio of (10-5): 1. In an elution process, the elution process may be monitored by thin-layer chromatography (TLC), and the ratio of the eluent may be adjusted based on a separation of a product. After the solvent is removed from the eluate by rotary evaporation, a target product is obtained.

Preferably, the solvothermal reaction includes the following steps: The imidazole-functionalized benzene tricarboxylic acid organic ligand and a salt containing the nodal metal are dissolved in N,N-dimethylformamide, sealed, allowed to react at 120° C.-140° C. for 48-96 h, and cooled to room temperature, and solids are collected. The collected solids (crystals) are washed several times with a washing solvent and then heated and dried under a vacuum condition to remove solvent molecules and unreacted small molecules blocked in pores, and ultimately, an Im@RE-MOF porous crystal material with a lipase-mimetic catalytic activity is obtained. In this step, the washing solvent is DMF and anhydrous ethanol, the washing frequency is 3-5 times, the solvent exchange time is 6 hours to 12 hours for each time of washing, the temperature for drying is 120° C. to 150° C., and the drying time is 8 hours to 16 hours.

Preferably, in the solvothermal reaction, a molar ratio of the imidazole-functionalized benzene tricarboxylic acid organic ligand to the salt containing the nodal metal is 1: (1-2), a ratio of the salt containing the nodal metal to an organic solvent is (0.5-2) mmol: (20-50) mL, and the organic solvent is N, N-dimethylformamide.

Preferably, the salt containing the nodal metal is ZrCl₄, Sc(NO₃)₃·xH₂O, or Cd(NO₃)₂·4H₂O.

The imidazole-functionalized metal-organic framework material Im@RE-MOF, prepared by the aforementioned preparation method, is used in the hydrolysis or synthesis of ester compounds.

Preferably, the ester compounds are carboxylic esters.

Preferably, in catalyzing hydrolysis of the ester compounds, the nodal metal is Zr, the reaction medium is a buffer solution with pH=7.0, the reaction time is 30 minutes to 1 hour, and the reaction temperature is room temperature (20° C.-30° C.).

Preferably, in the hydrolysis of the ester compounds, a ratio of an amount of the ester compounds used to an amount of the imidazole-functionalized metal-organic framework material Im@RE-MOF used is 1 mmol to 20 mg.

Preferably, in the synthesis of the ester compounds, the nodal metal is Sc or Cd, the reaction system is solvent-free, the reaction time is 6-12 h, and the reaction temperature is room temperature (20° C.-30° C.).

Preferably, in the synthesis of the ester compounds, a ratio of a carboxylic acid substrate to the imidazole-functionalized metal-organic framework material Im@RE-MOF is 1 mmol: (45-60) mg.

### Beneficial Effects

Compared with the prior art, the present disclosure has the following significant advantages:

1. Excellent stability and reusability: Compared to natural protein enzymes, the MOF material in the present disclosure has a rigid crystalline framework, exhibits higher thermal stability and chemical stability, and, as a heterogeneous catalyst, is easily separated from a reaction system by filtration or centrifugation.

2. Efficient biomimetic catalytic activity: The Im@RE-MOF material prepared in the present disclosure successfully mimics a catalytic function of lipase and exhibits excellent catalytic activity for ester hydrolysis and ester synthesis under mild conditions. For catalytic hydrolysis of p-nitrophenyl caproate, the catalytic ability of the Im@RE-MOF is comparable to that of natural lipase, achieving a reaction rate of 0.8 mmol to 1.2 mmol of substrate per minute. For the synthesis of ester compounds, a substrate conversion rate can reach 85% to 98%.

3. Wide application prospects: The material provides a new, efficient, and stable catalyst option for green synthesis of chiral pharmaceutical intermediates and fine chemicals and has great potential for industrial applications.

### BRIEF DESCRIPTION OF FIGURES

FIG. 1 shows an ESI mass spectrum of an imidazole ring-modified mesitylene intermediate product;

FIG. 2 shows a 1HNMR (nuclear magnetic resonance, NMR) spectrum of an imidazole ring-modified mesitylene intermediate product (using deuterated chloroform as a solvent);

FIG. 3 shows a 13CNMR spectrum of an imidazole ring-modified mesitylene intermediate product (using deuterated chloroform as a solvent);

FIG. 4A shows XPS survey spectra of Im@Zr-MOF prepared in Example 1;

FIG. 4B shows C1s scan of Im@Zr-MOF prepared in Example 1;

FIG. 4C shows N 1s scan of Im@Zr-MOF prepared in Example 1;

FIG. 4D shows O 1s scan of Im@Zr-MOF prepared in Example 1;

FIG. 4E shows Zr 3d scan of Im@Zr-MOF prepared in Example 1;

FIG. 5A shows XPS survey spectra of Im@Sc-MOF prepared in Example 2;

FIG. 5B shows C1s scan of Im@Sc-MOF prepared in Example 2;

FIG. 5C shows N 1s scan of Im@Sc-MOF prepared in Example 2;

FIG. 5D shows O 1s scan of Im@Sc-MOF prepared in Example 2;

FIG. 5E shows Zr 3d scan of Im@Sc-MOF prepared in Example 2;

FIG. 6A shows XPS survey spectra of Im@Cd-MOF prepared in Example 3;

FIG. 6B shows C1s scan of Im@Cd-MOF prepared in Example 3;

FIG. 6C shows N 1s scan of Im@Cd-MOF prepared in Example 3;

FIG. 6D shows O 1s scan of Im@Cd-MOF prepared in Example 3;

FIG. 6E shows Zr 3d scan of Im@Cd-MOF prepared in Example 3.

### DETAILED DESCRIPTION

The technical solutions of the present disclosure are further explained below in conjunction with examples.

### Example 1

### (1) Preparation of imidazole-4-acetyl chloride

First, imidazole-4-acetyl chloride was prepared. 13.5 g (about 0.107 mol) of imidazole-4-acetic acid was weighed, dissolved in 100 ml of anhydrous dichloromethane, and placed in a reaction bottle filled with nitrogen gas. Under a condition of a 0° C.-5° C. ice-water bath, 15.3 g (about 0.129 mol) of thionyl chloride was slowly added dropwise, with addition time controlled to be about 40 minutes. After the dropwise addition was completed, the reaction was continued under stirring at a temperature for 3 h. After the reaction was completed, a solvent was removed by rotary evaporation at 40° C. to obtain imidazole-4-acetyl chloride.

### (2) Preparation of imidazole-functionalized benzene tricarboxylic acid ligand

10 g (about 0.083 mol) of mesitylene and 17.8 g (about 0.134 mol) of aluminum chloride were dissolved in 100 ml of anhydrous dichloromethane. At 0° C.-5° C., the imidazole-4-acetyl chloride product prepared in step (1) was dissolved in 50 ml of anhydrous dichloromethane and then slowly added dropwise to the reaction system. After the dropwise addition was completed, the reaction was continued under stirring for 6 h. After the reaction was completed, a dilute hydrochloric acid solution (1 mol/L) was added for quenching. An organic phase was extracted by liquid separation, dried with anhydrous magnesium sulfate, filtered, and then subjected to rotary evaporation to obtain an imidazole ring-modified mesitylene intermediate product.

Then, an oxidation reaction was carried out. The resulting imidazole ring-modified mesitylene intermediate product was dissolved in 200 ml of nitric acid (70%), 4 g of potassium permanganate was added, and the reaction was carried out at 0° C.-10° C. for 18 h. After the reaction, the mixture was filtered, and a resulting crude product was purified by column chromatography on a silica gel column (60-200 meshes) using n-hexane and ethyl acetate (in a volume ratio of 8:1) as eluents. The eluate of a target product was collected, and the solvent was removed by rotary evaporation to obtain the imidazole-functionalized benzene tricarboxylic acid ligand.

### (3) Preparation of Im@RE-MOF

Then, 1 mmol of the imidazole-functionalized benzene tricarboxylic acid ligand and 1.5 mmol of zirconium chloride (ZrCl₄) were weighed and mixed in 30 mL of DMF. The mixture was placed in a hydrothermal reactor, heated to 130° C. at a rate of 5° C./min, and allowed to react for 72 h. After cooling, crystals were collected and washed three times with DMF, and solvent exchange was performed three times (by soaking for 8 h each time) with anhydrous ethanol. Finally, a product was vacuum dried at 150° C. for 12 h to obtain a white powder Im@Zr-MOF.

### (4) Enzyme-Mimetic Activity of Im@RE-MOF

The prepared Im@Zr-MOF was used for catalyzing a hydrolysis reaction of p-nitrophenyl caproate, and specific operations were as follows: 10 mg of the catalyst was weighed and added to 50 ml of a buffer solution (pH 7.0), 0.5 mmol of p-nitrophenyl caproate was dissolved, and the reaction was carried out under stirring at 25° C. for 30 minutes. After the reaction was completed, the concentration of p-nitrophenol produced was detected by a UV spectrophotometer, and the results showed that the catalytic efficiency was comparable to that of natural lipase. A reaction rate of 0.8 mmol of substrate per minute was achieved.

### Structural Characterization

1. The structural characterization included mass spectrometry and hydrogen and carbon nuclear magnetic resonance spectroscopy analyses of the imidazole ring-modified mesitylene intermediate product, which has the structural formula I.

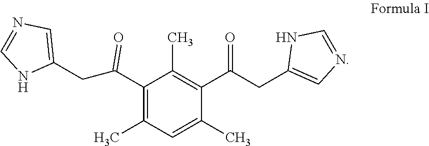 [SMILES: Cc1cc(C)c(C(=O)Cc2cnc[nH]2)c(C)c1C(=O)Cc1cnc[nH]1]

### 1,1′-(2,4,6-trimethyl-1,3-phenylene)bis(2-(1H-imidazol-5-yl)ethan-1-one)

A mass spectrum of the imidazole ring-modified mesitylene was obtained on a MALDI SYNAPT MS ultra-high performance liquid chromatography tandem quadrupole time-of-flight mass spectrometer from Waters Corporation (US).

Nuclear magnetic resonance hydrogen and carbon spectra of the imidazole ring-modified mesitylene were obtained on an Advance III 400 MHz fully digital nuclear magnetic resonance spectrometer (400 MHz) from Bruker AXS GmbH (Germany).

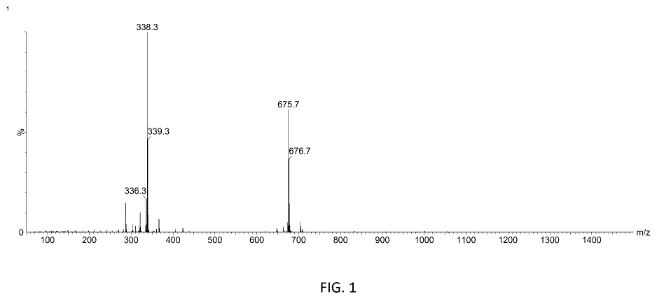

As shown in FIG. 1-FIG. 3, the 336.3 peak is prominent in the mass spectrum of FIG. 1, with the 338.3 peak, possibly corresponding to the [M+2H]⁺ ion, and the 339.3 peak being a +1 isotope peak, which conforms to a molecular ion peak and natural isotope distribution thereof of a double-substituted product. The 338.3 peak may be a molecular ion [M]⁺ or a hydrogenated product formed by slight hydrogenation (addition of 2H). The 339.3 peak is an [M+H]⁺ ion, i.e., a protonated molecular ion. The 675.7 peak is about twice of the peak 338, minus 1, which conforms to a mass-to-charge ratio of a dimer ion [M₂−H]⁺ or [M₂+H]⁺, and is commonly seen in ESI soft ionization where non-covalent bonds are formed between molecules.

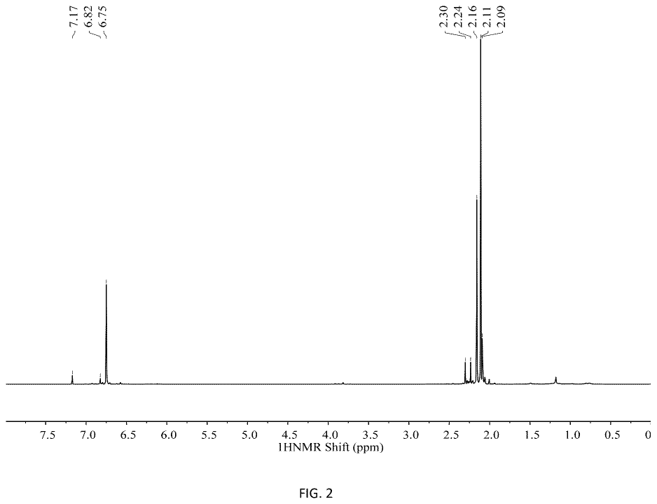

1H NMR (hydrogen spectrum) analysis is presented in FIG. 2: A chemical shift of 2.0-2.4 ppm is a proton signal of a methyl group (—CH₃) of trimethylbenzene. A chemical shift of 6.7-7.2 ppm is a signal of an aromatic ring proton (Ar—H).

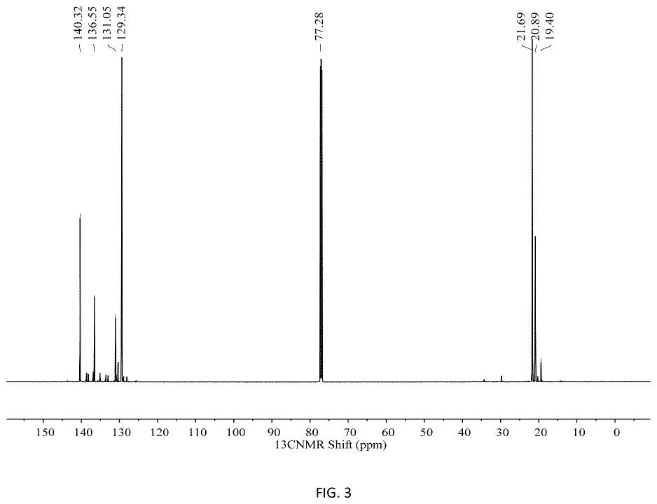

13C NMR (carbon spectrum) analysis is shown in FIG. 3: Multiple peaks of a chemical shift of 19-22 ppm belong to methyl carbon (—CH₃) in different chemical environments. A chemical shift of 77.28 ppm is the peak of a deuterated chloroform (CDCl₃) solvent. A chemical shift of 129-141 ppm belongs to aromatic ring carbon and double bond carbon.

According to the results of the mass spectrometry, hydrogen spectroscopy, and carbon spectroscopy, the imidazole ring-modified mesitylene intermediate product was successfully synthesized.

### 2. Survey Spectrum and High-Resolution Spectrum Analysis on Im@Zr-MOF

An XPS survey spectrum and a high-resolution spectrum were obtained on a K-Alpha X-ray photoelectron spectrometer (XPS) from Thermo Scientific (US). Test conditions: Excitation source: Al K α-ray (hv=1486.6 eV); Beam spot: 400 um; Vacuum degree of an analysis chamber: 5.0E-7 mBar; Working voltage: 12 kV; Filament current: 6 mA; Survey spectrum scan: pass energy of 150 eV and step size of 1 eV; and narrow spectrum scan: pass energy of 50 eV and step size of 0.1 eV, where at least 5 cycles of signal accumulation were performed for the narrow spectrum scan. Correction of binding energy: Charge correction was performed using a binding energy of C1s=284.80 eV as an energy standard.

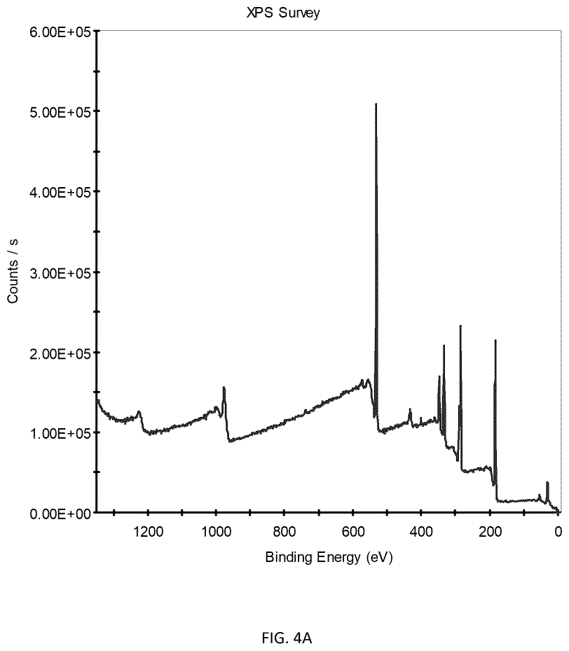

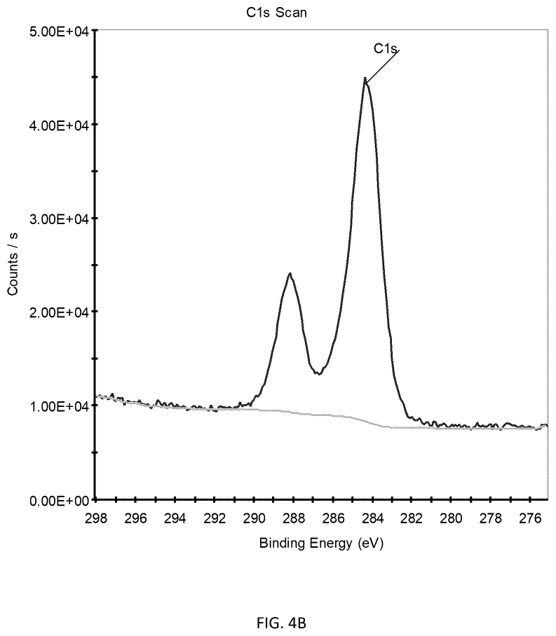

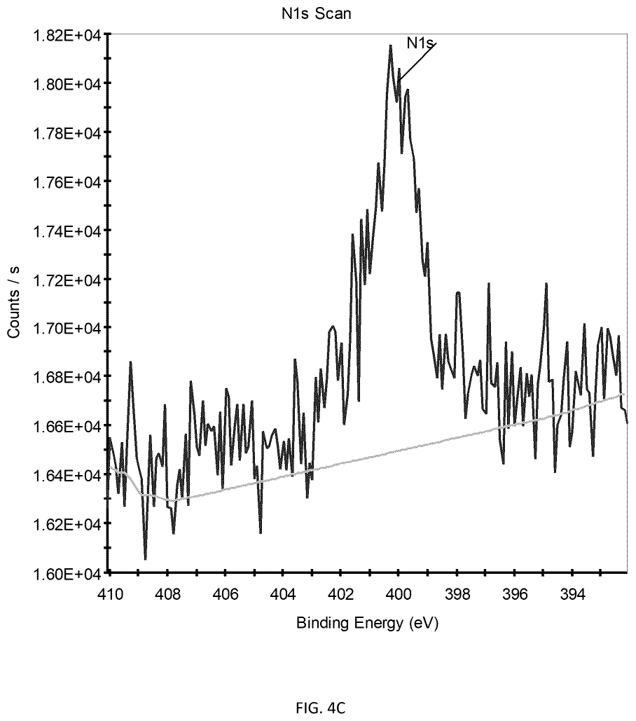

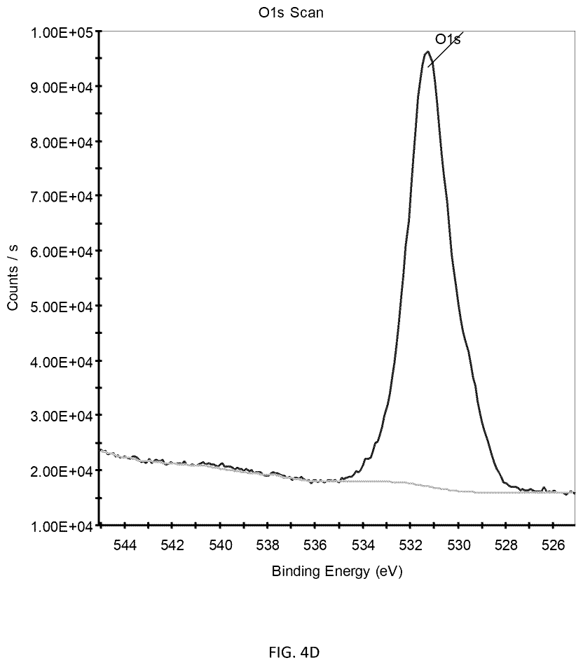

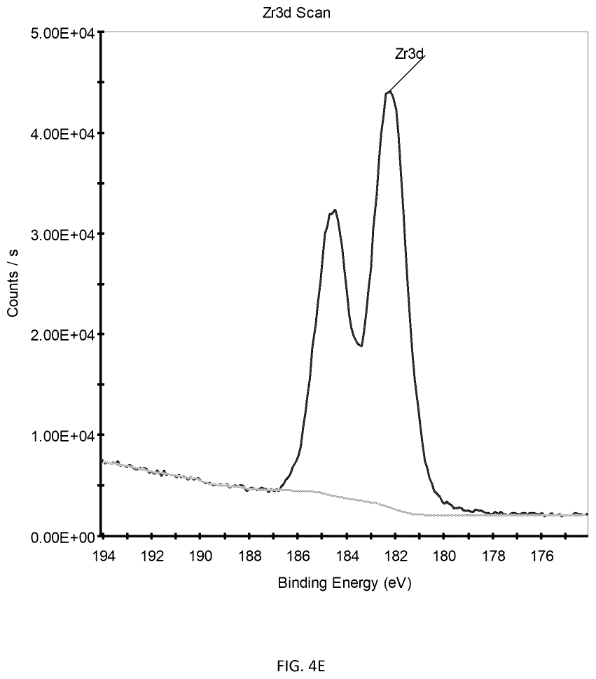

As shown in FIG. 4, the Zr 3d spectrum displays two clear peaks, located at about 182.2 eV (Zr 3d_{5/2}) and 184.5 eV (Zr 3d_{3/2}), respectively, which are the typical binding energies of +4 valent zirconium (Zr (IV)). The O 1s spectrum has a very strong and sharp peak at about 531.3 eV, which is the typical binding energy of oxygen in a carboxyl group (—COO⁻) or a carbonyl group (C═O). Also, the binding energy of Zr—O often appears in the region. The intensity of the peak is very high, indicating a high content of oxygen on the sample surface and a relatively homogeneous chemical environment.

The C1s spectrum clearly fits into two peaks. The main peak is located at about 284.8 eV, which is a standard C—C/C—H bond (from a benzene ring and an alkyl chain). The peak located at about 288.8 eV is a characteristic peak of a carbon atom (O—C═O) in a carboxyl group. The most reasonable explanation for the results of the C1s spectrum, O 1s spectrum, and Zr 3d spectrum is that a Zr (IV) ion coordinated with a carboxyl group of benzoic acid, forming a chemical bond of Zr—O—C. A low signal-to-noise ratio of the N 1s spectrum indicates that the nitrogen content on the sample surface is much lower than those of carbon and oxygen. In the noise background, a broad signal can be seen with a center at about 400.0 eV, which is the characteristic peak of uncoordinated imidazole nitrogen. This indicates that imidazole is not the primary ligand for Zr ions.

### Example 2

### (1) Preparation of imidazole-4-acetyl chloride

10.6 g (about 0.084 mol) of imidazole-4-acetic acid was weighed, dissolved in 80 ml of anhydrous dichloromethane, and placed in a reaction bottle filled with nitrogen gas. Under a condition of a 0° C.-5° C. ice-water bath, 12 g (about 0.101 mol) of thionyl chloride was slowly added dropwise, with addition time controlled to be about 30 minutes. After the dropwise addition was completed, the reaction was continued under stirring at the temperature for 3 h. After the reaction was completed, the solvent was removed by rotary evaporation at 40° C. to obtain imidazole-4-acetyl chloride.

### (2) Preparation of Imidazole-Functionalized Benzene Tricarboxylic Acid Ligand

8 g (about 0.067 mol) of mesitylene and 14 g (about 0.105 mol) of aluminum chloride were dissolved in 80 mL of anhydrous dichloromethane. At 0° C.-5° C., the imidazole-4-acetyl chloride product prepared in step (1) was dissolved in 40 mL of anhydrous dichloromethane and then slowly added dropwise to a reaction system. After the dropwise addition was completed, the reaction was continued under stirring for 5 h. After the reaction was completed, a dilute hydrochloric acid solution (1 mol/L) was added for quenching. An organic phase was extracted by liquid separation, dried with anhydrous magnesium sulfate, filtered, and then subjected to rotary evaporation to obtain an imidazole ring-modified mesitylene intermediate product.

Then, an oxidation reaction was carried out. The resulting imidazole ring-modified mesitylene intermediate product was dissolved in 150 ml of nitric acid (70%), 3 g of potassium permanganate was added, and the reaction was carried out at 0° C.-10° C. for 18 h. After the reaction, the mixture was filtered, and a resulting crude product was purified by column chromatography on a silica gel column (60-200 meshes) using n-hexane and ethyl acetate (in a volume ratio of 10:1) as eluents. The eluate of a target product was collected, and the solvent was removed by rotary evaporation to obtain a purified imidazole-functionalized benzene tricarboxylic acid ligand.

### (3) Preparation of Im@RE-MOF

0.8 mmol of the imidazole-functionalized benzene tricarboxylic acid ligand and 1.2 mmol of scandium nitrate (Sc(NO₃)₃·xH₂O) were weighed and mixed in 25 mL of DMF. The mixture was placed in a hydrothermal reactor, heated to 120° C. at a rate of 5° C./min, and allowed to react for 48 h. After cooling, crystals were collected and washed three times with DMF, and solvent exchange was performed three times (by soaking for 6 h each time) with anhydrous ethanol. Finally, a product was vacuum dried at 140° C. for 10 h to obtain a white powder Im@Sc-MOF.

### (4) Enzyme-Mimetic Activity of Im@RE-MOF

The prepared Im@Sc-MOF was used for catalyzing an ester synthesis reaction of piperidine-3-carboxylic acid and ethanol, and specific operations were as follows: 15 mg of the catalyst was weighed and added to 50 ml of an ethanol solvent, 0.3 mmol of piperidine-3-carboxylic acid was added, and the reaction was carried out under stirring at 25° C. for 8 h. After the reaction was completed, the purity of the product ethyl piperidine-3-carboxylate was measured by liquid chromatography, and the results showed that the substrate conversion rate reached 95%.

### Structural Characterization

### Characterization of Im@Sc-MOF:

An XPS survey spectrum and a high-resolution spectrum were obtained on a K-Alpha X-ray photoelectron spectrometer (XPS) from Thermo Scientific (US). Test conditions: Excitation source: Al K α-ray (hv=1486.6 eV); Beam spot: 400 μm; Vacuum degree of an analysis chamber: 5.0E-7 mBar; Working voltage: 12 kV; Filament current: 6 mA; Survey spectrum scan: pass energy of 150 eV and step size of 1 eV; and narrow spectrum scan: pass energy of 50 eV and step size of 0.1 eV, where at least 5 cycles of signal accumulation were performed for the narrow spectrum scan. Correction of binding energy: Charge correction was performed using a binding energy of C1s=284.80 eV as an energy standard.

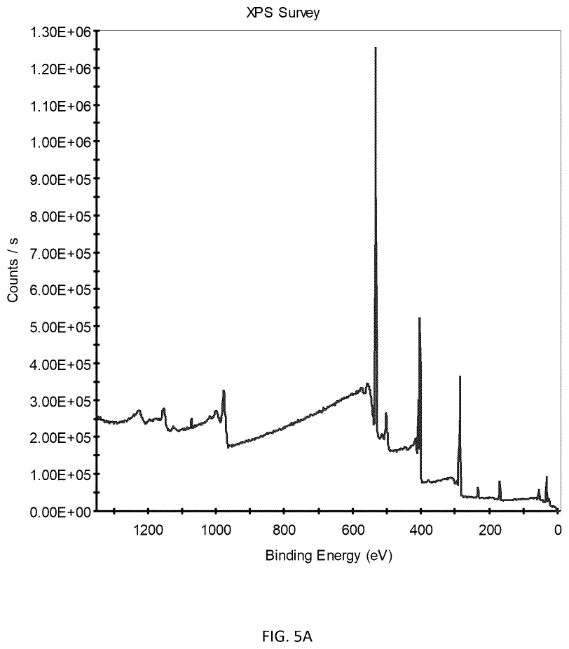

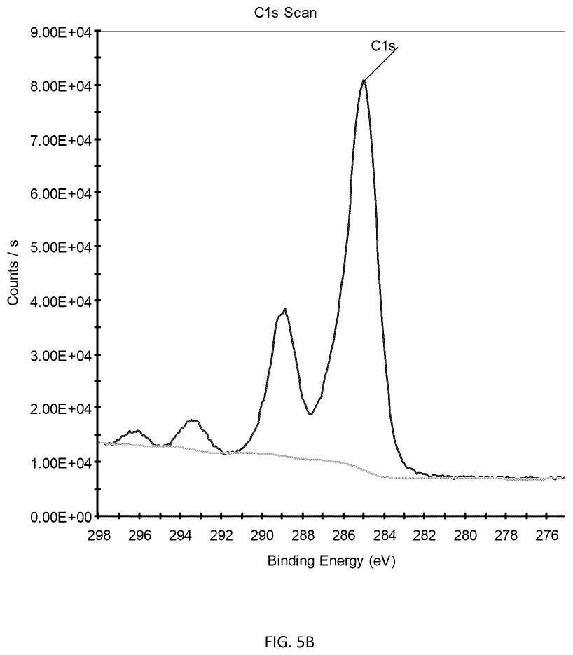

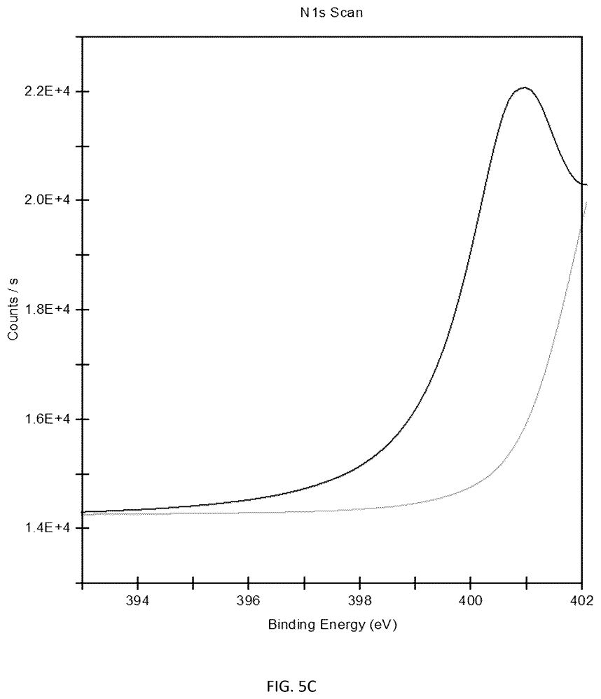

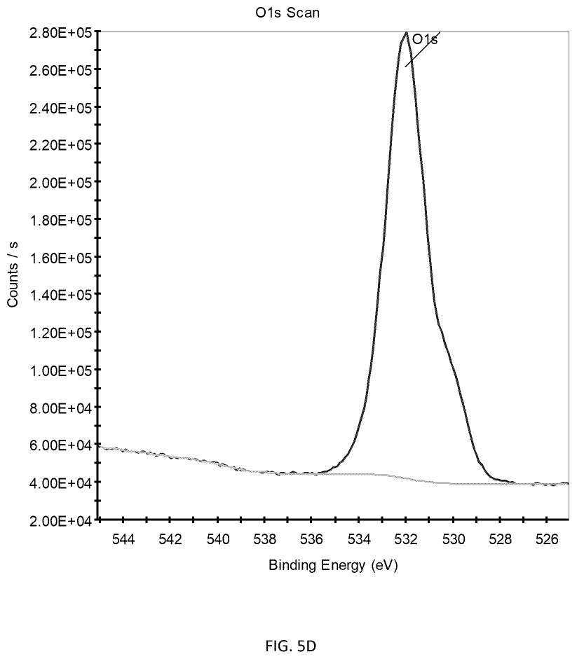

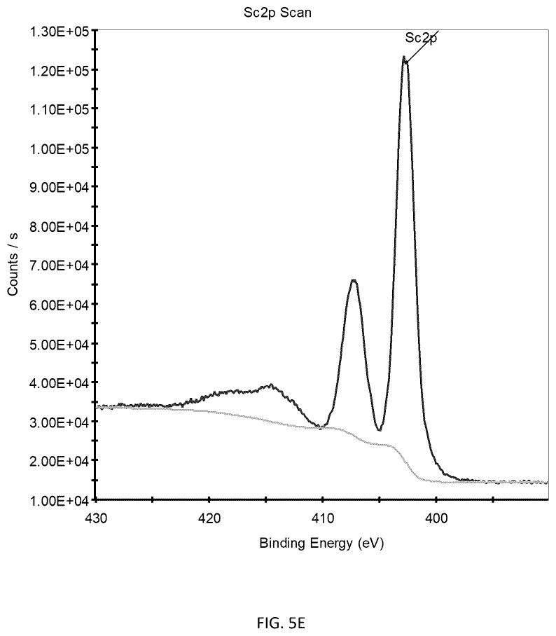

As shown in FIG. 5, the Sc 2p spectrum displays two peaks: One strong peak is located at about 402.2 eV (Sc 2p3/2), and the other weak peak is located at about 407.0 eV (Sc 2p1/2). The two peaks are typical spin orbit-splitting peaks of the Sc element, and the peak position and energy difference (about 4.8 eV) belong to Sc(III) (+3 valent scandium). The N 1s spectrum displays a peak with a peak top at about 401 eV. The O 1s spectrum exhibits a very sharp and symmetrical single peak at about 531.6 eV. The single peak is a typical characteristic of coordination between a carboxylate radical (—COO⁻) and a metal ion. When a carboxyl group is deprotonated and coordinates with Sc³⁺, the chemical environment of the two oxygen atoms becomes equivalent (Sc—O—C—O—Sc), and thus only one peak appears in XPS.

### Example 3

### (1) Preparation of Imidazole-4-Acetyl Chloride

16.8 g (about 0.133 mol) of imidazole-4-acetic acid was weighed, dissolved in 120 ml of anhydrous dichloromethane, and placed in a reaction bottle filled with nitrogen gas. Under a condition of a 0° C.-5° C. ice-water bath, 19 g (about 0.16 mol) of thionyl chloride was slowly added dropwise, with addition time controlled to be about 50 minutes. After the dropwise addition was completed, the reaction was continued under stirring at the temperature for 3 h. After the reaction was completed, the solvent was removed by rotary evaporation at 40° C. to obtain imidazole-4-acetyl chloride.

### (2) Preparation of Imidazole-Functionalized Benzene Tricarboxylic Acid Ligand

12 g (about 0.1 mol) of mesitylene and 22.2 g (about 0.167 mol) of aluminum chloride were dissolved in 120 mL of anhydrous dichloromethane. At 0° C.-5° C., the imidazole-4-acetyl chloride product prepared in step (1) was dissolved in 60 ml of anhydrous dichloromethane and then slowly added dropwise to a reaction system. After the dropwise addition was completed, the reaction was continued under stirring for 6 h. After the reaction was completed, a dilute hydrochloric acid solution (1 mol/L) was added for quenching. An organic phase was extracted by liquid separation, dried with anhydrous magnesium sulfate, filtered, and then subjected to rotary evaporation to obtain an imidazole ring-modified mesitylene intermediate product.

Then, the resulting imidazole ring-modified mesitylene intermediate product was dissolved in 250 mL of nitric acid (70%), 5 g of potassium permanganate was added, and the reaction was carried out at 0° C.-10° C. for 18 h. After the reaction, the mixture was filtered, and a resulting crude product was purified by column chromatography on a silica gel column (60-200 meshes) using n-hexane and ethyl acetate (in a volume ratio of 5:1) as eluents. The eluate of a target product was collected, and the solvent was removed by rotary evaporation to obtain a purified imidazole-functionalized benzene tricarboxylic acid ligand.

### (3) Preparation of Im@RE-MOF

1.2 mmol of the imidazole-functionalized benzene tricarboxylic acid ligand and 1.8 mmol of cadmium nitrate (Cd(NO₃)₂·4H₂O) were weighed and mixed in 40 ml of DMF. The mixture was placed in a hydrothermal reactor, heated to 140° C. at a rate of 5° C./min, and allowed to react for 96 h. After cooling, crystals were collected and washed three times with DMF, and solvent exchange was performed three times (by soaking for 12 h each time) with anhydrous ethanol. Finally, a product was vacuum dried at 150° C. for 16 h to obtain a white powder Im@Cd-MOF.

### (4) Enzyme-Mimetic Activity of Im@RE-MOF

The prepared Im@Cd-MOF was used for catalyzing an ester synthesis reaction of L-proline and ethanol, and specific operations were as follows: 20 mg of the catalyst was weighed and added to 50 ml of an ethanol solvent, 0.4 mmol of L-proline was added, and the reaction was carried out under stirring at 25° C. for 12 h. After the reaction was completed, the purity of L-proline ethyl ester was measured by liquid chromatography, and the results showed that the substrate conversion rate reached 98%.

### Structural Characterization

### Characterization of Im@Cd-MOF

An XPS survey spectrum and a high-resolution spectrum were obtained on a K-Alpha X-ray photoelectron spectrometer (XPS) from Thermo Scientific (US). Test conditions: Excitation source: Al K α-ray (hv=1486.6 eV); Beam spot: 400 μm; Vacuum degree of an analysis chamber: 5.0E-7 mBar; Working voltage: 12 kV; Filament current: 6 mA; Survey spectrum scan: pass energy of 150 eV and step size of 1 eV; and narrow spectrum scan: pass energy of 50 eV and step size of 0.1 eV, where at least 5 cycles of signal accumulation were performed for the narrow spectrum scan. Correction of binding energy: Charge correction was performed using a binding energy of C1s=284.80 eV as an energy standard.

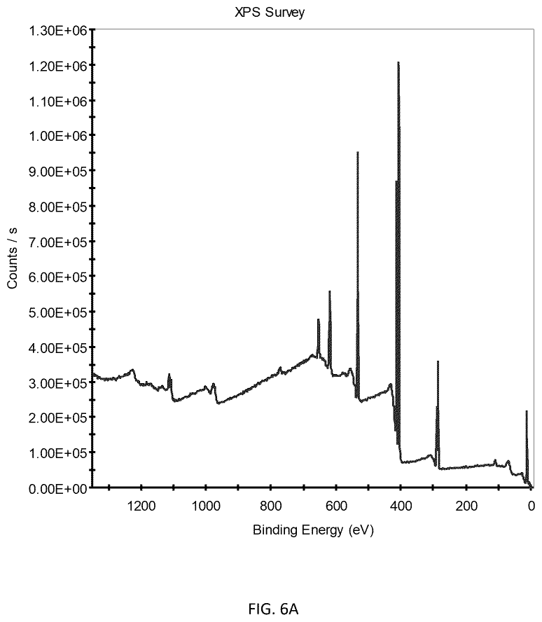

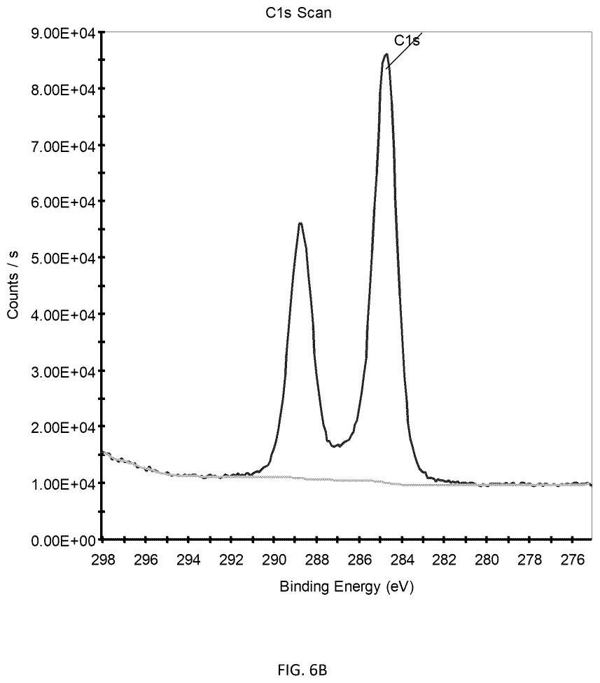

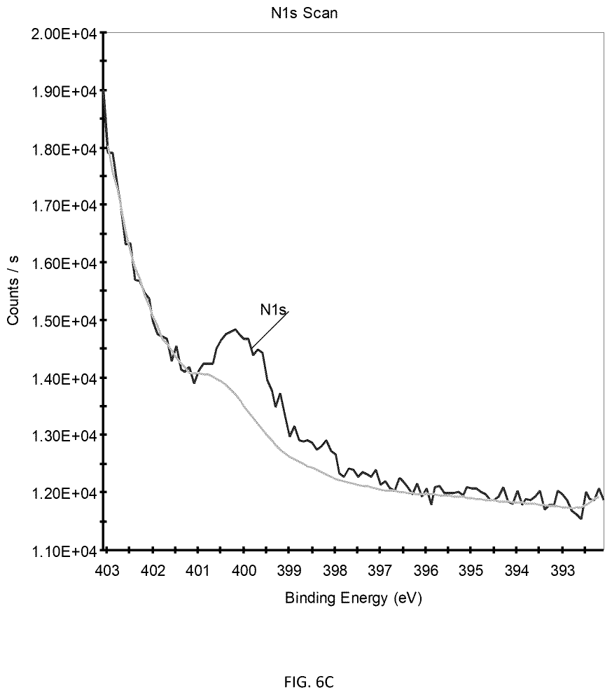

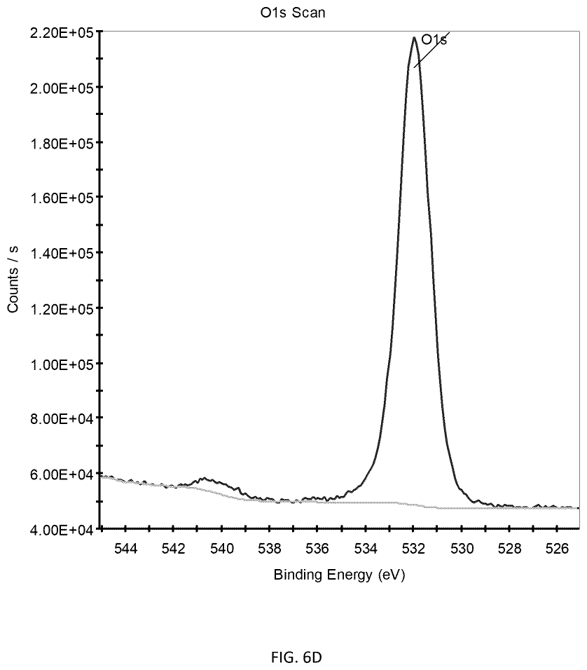

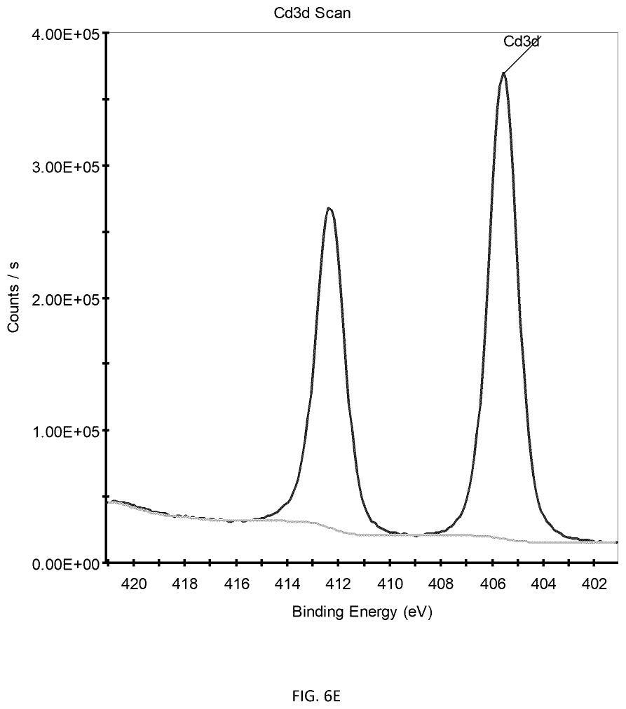

As shown in FIG. 6, the Cd 3d high-resolution spectrum displays two sharp peaks, namely Cd 3d_{5/2} (about 405.5 eV) and Cd 3d_{3/2} (about 412.2 eV), which are typical spin orbit splitting peaks, indicating that the Cd element exists in the form of +2 valence (Cd²⁺). The O 1s high-resolution spectrum exhibits a very sharp and symmetrical single peak at about 532 eV, which is a typical characteristic of successful coordination between a carboxyl group and a metal ion. When the carboxyl group is deprotonated and coordinates with a metal ion (e.g., Cd²⁺), α-COO⁻—Cd structure is formed, and the chemical environment of the two oxygen atoms becomes very similar. Therefore, the O 1s spectrum changes from a wider peak or two peaks fitted together into a single peak. The strong peak at ˜284.8 eV in the C1s high-resolution spectrum is a C—C/C—H bond in benzene rings and imidazole rings, as well as contaminated carbon used as a calibration standard. The peak at ˜288.5 eV corresponds to the typical binding energy of carbon (O═C—O) in a carboxyl group after coordination. A low signal-to-noise ratio of the N 1s spectrum indicates that the nitrogen content on the sample surface is much lower than those of carbon and oxygen.

### Comparative Example 1

### (1) Preparation of Zr-MOF Substrate Material

1 mmol of benzene tricarboxylic acid (H₃BTC, i.e., a basic ligand that has not been imidazole functionalized yet) and 1.5 mmol of zirconium chloride (ZrCl₄) were weighed and mixed in 30 ml of N,N-dimethylformamide (DMF). The mixture was placed in a hydrothermal reactor, heated to 130° C. at a rate of 5° C./min, and allowed to react for 72 h at the temperature. After the reaction was completed, the mixture was allowed to cool naturally to room temperature, the crystals produced were collected and washed three times with DMF, and solvent exchange was then performed three times (by soaking for 8 h each time) with anhydrous ethanol. Finally, a sample was vacuum dried at 50° C. for 12 h to obtain a white powder Zr-MOF substrate material (MOF-808).

The structure of the material is similar to the Im@Zr-MOF in Example 1 but lacks a covalently linked imidazole catalytic group.

### (2) Catalytic Activity Test of Zr-MOF Material

The prepared Zr-MOF substrate material was used for catalyzing an ester synthesis reaction of piperidine-3-carboxylic acid and ethanol, and specific operations were as follows: 15 mg of a substrate Zr-MOF catalyst was weighed and added to 50 mL of an ethanol solvent, then 0.3 mmol of a piperidine-3-carboxylic acid substrate was added, and the reaction system was stirred magnetically at 25° C. for 8 h. After the reaction was completed, a sample was taken to measure the production of the product ethyl piperidine-3-carboxylate by high-performance liquid chromatography (HPLC). The results of liquid chromatography analysis showed that the substrate conversion rate was lower than 5%.

### Comparative Example 2

### (1) Preparation of Im@Al-MOF

Refer to Example 1 for preparation of the imidazole-functionalized benzene tricarboxylic acid ligand.

1 mmol of the imidazole-functionalized benzene tricarboxylic acid ligand and 1.5 mmol of aluminum nitrate trihydrate (Al (NO₃)₃·9H₂O) were weighed and mixed in 30 ml of N,N-dimethylformamide (DMF). The mixture was placed in a hydrothermal reactor, heated to 130° C. at a rate of 5° C./min, and allowed to react for 72 h at the temperature. After the reaction was completed, the mixture was allowed to cool, a solid product was collected, the product was washed three times with DMF, and solvent exchange was performed three times (by soaking for 8 h each time) with anhydrous ethanol. Finally, the product was vacuum dried at 50° C. for 12 h to obtain a powder product denoted as Im@Al-MOF.

### (2) Catalytic Activity Test of Im@Al-MOF

15 mg of an Im@Al-MOF catalyst was weighed and added to 50 ml of an ethanol solvent, then 0.3 mmol of a piperidine-3-carboxylic acid substrate was added, and a reaction system was stirred magnetically at 25° C. for 8 h. After the reaction was completed, a sample was taken to measure the production of the product ethyl piperidine-3-carboxylate by high-performance liquid chromatography (HPLC), and the substrate conversion rate was calculated as 7.2±1.2%.

### APPLICATION EXAMPLE

The catalytic performance of the Im@Zr-MOF, Im@Sc-MOF, and Im@Cd-MOF prepared in Examples 1, 2, and 3, as well as the Im@Al-MOF catalyst prepared in Comparative Example 2 in catalyzing an ester synthesis reaction of piperidine-3-carboxylic acid and ethanol was compared.

15 mg of a catalyst was weighed and added to 50 ml of an ethanol solvent, then 0.3 mmol of piperidine-3-carboxylic acid was added, and a reaction system was stirred magnetically at 25° C. for 8 h. After the reaction was completed, a sample was taken to measure the production of the product ethyl piperidine-3-carboxylate by high-performance liquid chromatography (HPLC), and the substrate conversion rate was calculated.

The results showed that the substrate conversion rates for synthesizing the ethyl piperidine-3-carboxylate using the Im@Zr-MOF, Im@Sc-MOF, Im@Cd-MOF, and Im@Al-MOF as catalysts were 95.2±2.5%, 92.6±1.3%, 75.6±3.5%, and 7.2±1.2%, respectively.

### Comparative Example 3

The only difference from Example 1 was that the oxidation reaction conditions in step (2) were adjusted. Specifically, the imidazole ring-modified mesitylene was dissolved in 200 ml of nitric acid (70%) and 4 g of potassium permanganate was added, and the reaction was performed under stirring at 80° C. to accelerate an oxidation reaction process. When heated to about 60° C., the reaction mixture began to boil violently, and after 8 h of continuous heating, the reaction solution turned into a dark brown turbid liquid. After the reaction solution cooled down, no target product, i.e., the imidazole-functionalized benzene tricarboxylic acid ligand, was detected by thin layer chromatography (TLC) and high-performance liquid chromatography (HPLC).

### Comparative Example 4

The only difference from Example 1 was that in a preparation process of the imidazole ring-modified mesitylene intermediate product in step (2), 5.0 g (about 0.042 mol) of mesitylene and 17.8 g (about 0.134 mol) of aluminum chloride were dissolved in 100 ml of anhydrous tetrahydrofuran (THF); at 0° C.-5° C., all the imidazole-4-acetyl chloride product (about 0.107 mol) prepared in step (1) was dissolved in 50 ml of anhydrous THF and then slowly added dropwise to a reaction system; after the dropwise addition was completed, the reaction was continued under stirring for 6 h at the temperature; after the reaction was completed, dilute hydrochloric acid was added for quenching according to the method of Example 1; and an organic phase was extracted by liquid separation. A residue obtained after the organic phase was evaporated was analyzed by high performance liquid chromatography (HPLC) and hydrogen nuclear magnetic resonance spectroscopy (1H NMR). The results showed that the main components were unreacted mesitylene and the hydrolysis product (imidazole-4-acetic acid) of the imidazole-4-acetyl chloride, while the yield of the target product, the “imidazole ring-modified mesitylene intermediate product”, was less than 1%, substantially indicating that the reaction did not occur.

The reason is that the catalyst aluminum chloride for the Friedel-Crafts acylation reaction is a strong Lewis acid, while tetrahydrofuran (THF) is a Lewis base, and the two form a stable acid-base complex (THF·AlCl₃). The complexation can “poison” the catalyst, causing it to lose its catalytic activity and preventing the acylation reaction from proceeding. Choosing the non-coordinating dichloromethane as the solvent successfully avoids the problem of catalyst deactivation and is the key to the success of the reaction.

The examples provided above are not intended to limit the scope covered by the present disclosure, nor are the steps described to limit the execution order. Apparent improvements made to the present disclosure by those skilled in the art based on existing common knowledge also fall within the scope of protection defined in the claims of the present disclosure.

What is claimed is:

1. A method for preparing an imidazole-functionalized metal-organic framework material, referred to as Im@RE-MOF, which exhibits lipase-mimetic catalytic activity, comprising the following steps:
(1) synthesizing imidazole-4-acetyl chloride from imidazole-4-acetic acid and a chlorinating agent as raw materials;
(2) carrying out a Friedel-Crafts acylation reaction on the imidazole-4-acetyl chloride and mesitylene as raw materials, with aluminum chloride as a catalyst, to obtain an imidazole ring-modified mesitylene intermediate;
(3) oxidizing all methyl groups of the imidazole ring-modified mesitylene intermediate to carboxyl groups with an oxidant to obtain an imidazole-functionalized benzene tricarboxylic acid organic ligand; and
(4) carrying out a solvothermal reaction on the imidazole-functionalized benzene tricarboxylic acid ligand and a nodal metal to obtain the imidazole-functionalized metal-organic framework material Im@RE-MOF.

2. The preparation method according to claim 1, comprising using at least one of the metals Zr, Sc, and Cd as the nodal metal.

3. The preparation method according to claim 1, wherein in step (1), the chlorinating agent is thionyl chloride or phosphorus trichloride, a molar ratio of the imidazole-4-acetic acid to the chlorinating agent is 1:(0.8-1.1), a reaction solvent is anhydrous dichloromethane, a reaction temperature is 0° C.-5° C., and the reaction time is 2-3 hours.

4. The preparation method according to claim 1, wherein in step (2), a molar ratio of the imidazole-4-acetyl chloride to the mesitylene is 1:(0.8-1.1), an amount of the catalyst used is 1.5 times the molar number of the imidazole-4-acetyl chloride, a reaction solvent is anhydrous dichloromethane, a reaction temperature is 0° C.-5° C., and a reaction time is 4-6 hours.

5. The preparation method is according to claim 1, wherein in step (3), the oxidant is potassium permanganate, a reaction solvent is nitric acid, a reaction temperature is 0° C.-10° C., and reaction time is 6-18 hours.

6. The preparation method according to claim 1, wherein the solvothermal reaction comprises the following steps: the imidazole-functionalized benzene tricarboxylic acid organic ligand and a salt containing the nodal metal are dissolved in N,N-dimethylformamide, sealed, allowed to react at 120° C.-140° C. for 48-96 hours, and cooled, and solids are collected, a molar ratio of the imidazole-functionalized benzene tricarboxylic acid organic ligand to the salt containing the nodal metal being 1: (1-2), and a ratio of the salt containing the nodal metal to the N,N-dimethylformamide being (0.5-2) mmol: (20-50) mL.

7. A method for hydrolyzing or synthesizing esters, comprising using the imidazole-functionalized metal-organic framework material Im@RE-MOF prepared by the method according to claim 1, as a catalyst for the reaction.

8. The method according to claim 7, wherein in catalyzing hydrolysis of the ester compounds, the nodal metal is Zr, a ratio of an amount of the ester compounds used to an amount of the imidazole-functionalized metal-organic framework material Im@RE-MOF used is 1 mmol: (15-25) mg, a reaction medium is a buffer solution with pH=7.0, and the reaction is carried out at room temperature for 30-60 minutes.

9. The method according to claim 7, wherein in catalyzing synthesis of the ester compounds, the nodal metal is Sc or Cd, a ratio of an amount of a carboxylic acid substrate used to an amount of the imidazole-functionalized metal-organic framework material Im@RE-MOF used is 1 mmol: (40-60) mg, and the reaction is carried out at room temperature for 6-12 hours.
